(19) United States
(12) Patent Application Publication
(10) Pub. No.: US 2001/0047144 A1
(43) Pub. Date: Nov. 29, 2001

(54) Luminescent medical bandage

(76) Inventors: Scott Andrew Tillotson, North Kingstown, RI (US); Paul Brian Murphy, Somerville, MA (US)

Correspondence Address: HARRINGTON & SMITH, LLP, 1809 BLACK ROCK TURNPIKE, FAIRFIELD, CT, 06432, US

(21) Appl. No.: 09/793,694

(22) Filed: Feb. 26, 2001

Related U.S. Application Data
(60) Provisional application No. 60/185,275, filed on Feb. 29, 2000.

(51) Int. Cl. A61F 13/00

(52) U.S. Cl. 602/41

(57) ABSTRACT

A bandage includes at least one layer, where the layer further includes a light emitting material. The light emitting material may be evenly distributed throughout the layer, printed or coated on a surface of the layer, or may form an ornamental design on a surface of the layer. The light emitting material may also be printed as text on, or incorporated into the layer, thus allowing such text to be read in low light or dark conditions.

### FIELD OF THE INVENTION

The present invention relates to medical bandages, and in particular, to medical bandages that include at least one luminescent material that is phosphorescent or photoluminescent.

### BACKGROUND OF THE INVENTION

Phosphorescent or photoluminescent materials are luminescent, that is, they are capable of producing light. While phosphorescent materials may also be light reflectors, their primary function is to act as a source of light. Phosphorescent materials typically operate by absorbing a range of radiation wavelengths, converting this radiation to radiation in the visible spectrum and emitting it as light, visible to the eye. Phosphorescent materials are those materials that are capable of producing radiation in the visible spectrum for a period of time after the initial absorption of radiation has stopped. This phenomenon is generally recognized as a “glow in the dark” characteristic of the material. For the purposes of this invention, luminescent, photoluminescent, fluorescent, or phosphorescent materials in any combination are referred to herein as “light emitting materials”.

It is known to use “light emitting” or “glow in the dark” materials on various types of objects. These applications generally relate to the use of these materials to improve safety or for locating an object in the dark.

Reference in this regard, for example, may be had to U.S. Pat. No. 5,279,058, entitled “Phosphorescent Identification Device,” issued Jan. 18, 1994, and to U.S. Pat. No. 5,716,723, entitled “Glow In The Dark Shoe Sole,” issued Feb. 10, 1998.

Bandages of various types are well known in the art. A typical bandage may include a number of layers. These layers could include a cloth or plastic backing material with an outside surface for protecting the bandaged area, a pad layer, and a cushioning layer interposed between the backing material and the pad layer. For certain bandages the pad layer may be smaller than the backing material and the cushioning layer may be coated with an adhesive. The pad may be plastic-coated or otherwise treated to prevent the pad from adhering to a wound or bandaged area. The pad layer may also be treated with a substance to prevent infection or to provide some other treatments.

It is also known to provide images on medical bandages for novelty purposes, as shown in U.S. Design Patent No.: D408,540, entitled “Baseball Image On An Adhesive Bandage,” issued Apr. 20, 1999, and also shown in U.S. Design Patent No.: D410,446, entitled “Soccer Image On An Adhesive Bandage,” issued Jun. 1, 1999,

### OBJECTS OF THE INVENTION

It is a first object and advantage of this invention to provide an improved bandage including a light emitting material.

It is a further object and advantage of this invention to provide an improved bandage including a light emitting material as part of a substrate layer, or as part of a layer of material included in the bandage, or by being printed onto a surface of a layer of material included as a part of the bandage.

It is a further object and advantage of this invention to provide an improved bandage including at least one light emitting material for exhibiting improved visibility, especially in the dark, and/or for novelty use, and/or for safety reasons.

### SUMMARY OF THE INVENTION

A bandage is disclosed that includes at least one layer, where the layer further includes a light emitting material. The light emitting material may be evenly distributed throughout the layer, printed or coated on a surface of the layer, or it may form an ornamental design on a surface of the layer. The light emitting material may also be printed as text on the layer or may serve as a background for text, thus allowing such text to be read in low light or dark conditions.

### BRIEF DESCRIPTION OF THE DRAWINGS

FIG. 1 shows a side view of a bandage in accordance with the teachings of this invention; and

FIG. 2 shows a top view of the bandage.

### DESCRIPTION OF THE INVENTION

The objects and the advantages of the invention are realized by methods and apparatus in accordance with embodiments of this invention.

In one aspect, this invention provides for the use of light emitting materials as part of a bandage. As an example, the bandage may be a medical bandage including an adhesive for fastening the bandage to area to be treated or protected from further injury.

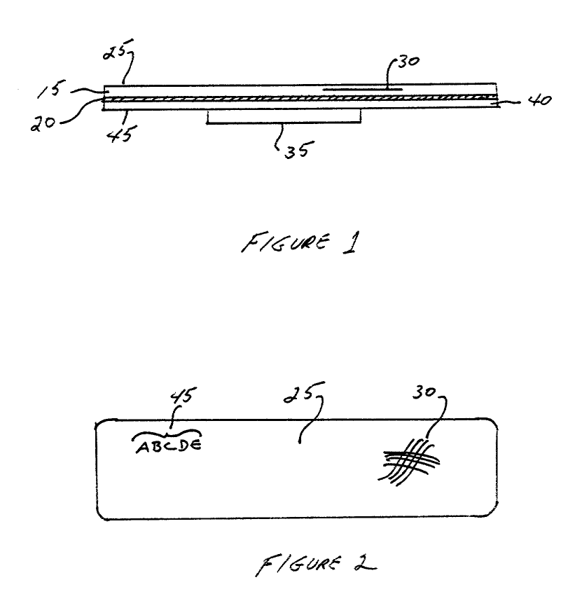

FIGS. 1 and 2 show examples of a bandage 10 in accordance with the teachings of the invention. The bandage 10 is shown to have a first layer 15. The first layer 15 preferably includes a backing material 20 which may be made from cloth or plastic. The first layer may also include an outside surface 25 for protecting the bandaged area.

One aspect of the invention provides for the use of a light emitting material 30 as a design element or as a coating on the outside surface 25 of the bandage 10, as shown in FIG. 2. The light emitting material 30 may be included continuously within the outside surface 25, or it may be printed on, or it may be coated on, the outside surface 25, either covering the entire outside surface 25, or covering at least a portion of the outside surface 25, for example, as an ornamental design. The light emitting material 30 may be incorporated into the outside surface 25 or in the first layer 15 as text 45 which could be readable in low light conditions or in total darkness.

An embodiment is also contemplated where the light emitting material 30 may be incorporated into the first layer 15, as shown in FIG. 1. The light emitting material 30 may be included continuously, or in an evenly dispersed fashion throughout the first layer 15, or it may be selectively printed on, or coated as part of the first layer 15. The light emitting material 30 may also be incorporated as part of an ornamental design into the first layer 15.

In another embodiment, the light emitting material 30 may be included as part of the backing material 20. As with the previously mentioned embodiments, the light emitting material 30 may be evenly dispersed throughout the backing material 20, or it may be selectively printed on, or coated on as part of a surface of the backing material 20, for example, as an ornamental design.

The bandage 10 may also include a pad 35 and a cushioning layer 40 interposed between the backing material 20 and the pad 35. For certain bandages the pad 35 may be smaller in area than the backing material 20 or the first layer 15. The cushioning layer 40 may be coated with an adhesive 45 on the surface facing the pad 35.

The light emitting material may be a derivative of a high persistence phosphor zinc sulfide compound, for example, ZnS:Cu. An example of a commercially available light emitting material would be Phosphorescent Pigment 2330 LBY available from USR Optonix, Inc. This material has a green emission color and is excitable by longwave, ultraviolet light. After a Xenon excitation for 6 minutes at 1000 lux, the emission from the light emitting material decays to 32 mcd/m² in approximately 10 minutes, and to 3.5 mcd/m² in approximately 60 minutes. This material may be mixed into any clear resin as a high load dispersion, or may be added directly and compounded into a resin of choice. In this case the resin containing the light emitting material is added to one or more layers of the bandage 10, as described above.

Another example of a light emitting material may include an inorganic luminescent material such as luminescent calcium sulfite, also called Canton's phosphor. This material may be made by igniting a mixture of calcium carbonate and sulfur with very small quantities of bismuth or manganese salts. Typical applications for luminescent calcium sulfite include luminous paints or varnishes.

Multiple light emitting materials, each emitting a different color, can also be employed in one bandage.

It should be understood that while the bandage has been described in a medical context that other applications are also contemplated, such as bandaging for industrial uses, for example, to effect a repair for piping, sheet metal, or other suitable industrial applications. Other industrial uses may include marking for targeting or avoidance in low light or dark conditions. For example, a luminescent bandage may be used to mark a valve so that the valve is visible in the event of a power failure.

It should be further understood that the descriptions of the light emitting materials and the structure of the bandages are presented as examples only, and that a wide variety of materials may be used to produce the desired luminescent, photoluminescent, fluorescent, or phosphorescent characteristics for bandages having any number of configurations. Furthermore, the bandage may have any desired shape, such as ovoid or circular or square.

Thus, while the invention has been particularly shown and described with respect to preferred embodiments thereof, it will be understood by those skilled in the art that changes in form and details may be made therein without departing from the scope and spirit of the invention.

1. A bandage comprising at least one layer, said at least one layer comprising a light emitting material.

2. A bandage according to claim 1, wherein said light emitting material is evenly distributed throughout said layer.

3. A bandage according to claim 1, wherein said light emitting material is printed on a surface of said layer.

4. A bandage according to claim 1, wherein said light emitting material is coated on a surface of said layer.

5. A bandage according to claim 1, wherein said light emitting material is used to form an ornamental design on a surface of said layer.

6. A bandage according to claim 1, wherein said light emitting material is used to form text on a surface of said layer.

7. A bandage according to claim 1, wherein said light emitting material is used to form text within said layer.

8. A bandage according to claim 1, wherein said light emitting material is incorporated into a backing material of said bandage.

9. A bandage according to claim 1, wherein said bandage is a medical bandage.

10. A bandage according to claim 1, further comprising an adhesive for fastening said bandage to an area to be covered by said bandage.

11. A bandage according to claim 1, wherein said light emitting material comprises a fluorescent or a phosphorescent compound.

12. A bandage according to claim 1, wherein said light emitting material comprises a high persistence phosphor zinc sulfide compound.

13. A bandage according to claim 1, wherein said light emitting material comprises luminescent calcium sulfite
